(19) United States
(12) Patent Application Publication
(10) Pub. No.: US 2013/0224157 A1
(43) Pub. Date: Aug. 29, 2013

(54) STEM CELLS FOR TREATING LUNG DISEASES

(71) Applicants: John J. Akabutu, Edmonton (CA); Bernard Thebaud, Edmonton (CA)

(72) Inventors: John J. Akabutu, Edmonton (CA); Bernard Thebaud, Edmonton (CA)

(21) Appl. No.: 13/624,729

(22) Filed: Sep. 21, 2012

Related U.S. Application Data
(63) Continuation of application No. 12/298,695, filed on Oct. 27, 2008, now abandoned.
(60) Provisional application No. 60/745,848, filed on Apr. 27, 2006.

(51) Int. Cl. A61K 35/14; A61K 38/18

(52) U.S. Cl. CPC A61K 35/14; A61K 38/1841; A61K 38/1875
USPC 424/93.7

(57) ABSTRACT

The invention is compositions and methods for treating lung diseases and conditions using mesenchymal stem cells. The preferred stem cells are those derived from a human umbilical cord, or from bone marrow.

### I. FIELD OF THE INVENTION

This invention relates to the use of stem cells to treat debilitating and life-threatening lung diseases that currently lack efficient therapies.

### II. BACKGROUND OF THE INVENTION

Preterm delivery is a major health care problem, affecting 10% of all births and accounting for more than 85% of all perinatal complications and death, Survival of extremely premature newborns (those born at less than about 28 weeks of gestation) has increased because of improvements in perinatal care, These infants however, are at high risk for long-term injury to both lung and brain.

Each year, 5000-10,000 newborns suffer from bronchopulmonary dysplasia (BPD), a chronic lung disease that follows ventilator and oxygen therapy for acute respiratory failure after premature birth. BPD has long term respiratory and neuro-developmental consequences that reach beyond childhood and result in increased health care costs. Further advances are required to increase survival free of neonatal morbidity or neuro-developmental impairment.

Emphysema, defined as airspace enlargement distal to terminal bronchioles, is a major component of chronic obstructive pulmonary disease (COPD), the fourth leading cause of death in the US. BPD and emphysema are characterized by interrupted development and loss of alveolar structures, and therapy is palliative. Other lung diseases that currently lack specific treatments and susceptible of benefiting from stem cell strategies include other causes of COPD, Cystic Fibrosis, Fibrosis, Acute Respiratory Distress Syndrome (ARDS), Pulmonary hypoplasia and Pulmonary Hypertension. Various animal models of these diseases are currently available in the laboratory or can be generated.

There are many reports suggesting a therapeutic potential for stem cells to prevent/repair heart, brain, liver and pancreatic (diabetes) diseases. There are several reports suggesting that bone-marrow derived stem cells can migrate to the intact or injured lung, but no therapeutic benefit has been shown so far to treat this organ, with the exception of intravenously administered endothelial progenitor cells to treat experimental pulmonary hypertension in rats. Also, no attempt to administer stem cells via the airways has been made so far. Very little is known about the resident lung stem cells (progenitor cells). Such cells reside in the trachea in the submucous gland duct and in the bronchi (as a subset of Clara-cell-specific expressing cells). Putative progenitor cells in the alveoli include the alveolar type 2 cells and Side populations (SP cells), each a potential avenue for isolation and treatment.

The use of mesenchymal cells, and mesenchymal cells culture in transplantation has been investigated. Using NOD/SCID mice, Noort, et al have demonstrated that co-transplantation of mesenchymal cells isolated as non-hematopoietic cells from fetal lung CD34+ cells significantly enhanced the engraftment of hematopoietic stem cells (Noon et al Exp Hematol 2002; 30:870-78). Similarly, Maitra et al (Maitra, et al., Bone Marrow Transplant 2004, 33:597-604) have demonstrated the successful repopulation of NOD/SCID mice with limited numbers of hematopoietic stem cells, augmented by co-infusion with unrelated human mesenchymal stem cells. Significantly, no enhancement was observed with a co-infusion with mouse mesenchymal stem cells. Human mesenchymal stem cells culture has also been shown to support the ex-vivo propagation of CD34+ cells, in the absence of direct contact between the mesenchymal and hematopoietic cells in culture, and enhance transplantation. (Sumner, et al, Cytother 2001 3; 422a).

Therapeutic use of mesenchymal stem cells and stem cell culture has been investigated. Administration of expanded mesenchymal stem cell cultures has been proposed for treatment of articular disorders (US Patent Application No. 20040151703 to Ha, et al), Hurler syndrome and metachromatic leukodystrophy (Koc, et al, Bone Marrow Transplant. 2002; 30:215-22) and for connective tissue engraftment, as well as hematopoietic cell engraftment (U.S. Pat. No. 6,355,239, to Bruder et al). Koc, et al (J Clin Oncol, 2000, 18:307-16) reported the co-infusion of culture expanded autologous bone marrow mesenchymal stem cells (in DMEM and bovine fetal serum medium) and peripheral blood progenitor cells in 32 breast cancer patients after high dose chemotherapy, with no observed toxicity or reduced engraftment related to the mesenchymal cell administration. Infusion of allogeneic bone marrow derived mesenchymal stem cells for Hurler syndrome and metachromatic leukodystrophy (Koc, et al, Bone Marrow Transplant. 2002; 30:215-22) also indicated no toxicity and possible therapeutic value. Recently, Lazarus, et al (Biol. Blood Marrow Transplant. 2005; 11:389-98) reported the co-administration of culture expanded mesenchymal stem cells and hematopoietic stem cells from sib-matched donors to 46 hematopological malignancy patients following high dose chemotherapy, with no toxicity and an increased probability of successful transplant in the co-administered group.

### III. SUMMARY OF THE INVENTION

This invention provides compositions and methods for treating various lung diseases and conditions. In preferred embodiments of the invention, the compositions and methods comprise mesenchymal stem cells. The composition may also include one or more surfactants. Surfactants typically have clinical relevance, and may be lead to improved lung distribution, i.e. enhanced therapeutic effect,

The present invention shows for the first time a quantitative and statistically significant beneficial therapeutic effect of stem cells for the treatment of a lung disease. The compositions and methods of the present invention may be capable of regenerating already established lung injury.

### IV. BRIEF DESCRIPTION OF THE DRAWINGS

FIG. 1 shows that administering bone marrow derived stem cells prevents oxygen-induced lung injury.

FIG. 2 shows that administering human umbilical cord blood derived stem cells prevents oxygen-induced lung injury.

FIG. 3 shows that bone marrow derived stem cells improve survival rates.

FIG. 4 shows that bone marrow derived stem cells co-cultured with oxygen-injured lung cells differentiate into alveolar epithelial cells.

### V. DETAILED DESCRIPTION OF THE INVENTION

The present invention provides compositions and methods for treating various lung diseases and conditions using mesenchymal stem cells (endothelial). In preferred embodiments of the invention, the stem cells are niche (bone marrow) derived (stem cells). In most preferred embodiments of the invention, the engineered stem cells are derived from human umbilical cord blood, even more preferably autologous stem cells.

As used herein, engineered refers to processing the cells so that they become more resistant to injury or have the ability to deliver or produce genes of interest. These processing steps are well known to those skilled in the art, and include, but are not limited to introducing or overexpressing a gene or genes of interest, and viral or non-viral vector mediated stem cell gene delivery. The compositions and methods of the present invention may include engineered mesenchymal stem cells (MSC) and/or non-engineered MSC.

Mesenchymal cells originate from the mesodermal layer of embryonic cells during development, and are present in every organ (including subcutaneous tissue such as lung and liver; and mesenchymal tissue, such as bone, cartilage, fat, tendon, skeletal muscle, and the stroma of bone marrow).

In some embodiments of the present invention, the mesenchymal cells are the adherent cell population after between 48 hrs to 7 days of culture at a high density without cytokine stimulation or supplemental medium.

In some embodiments of the present invention, autologous stem cells are preferred because self-derived cells are typically better immunologically when regenerating/repairing tissue and for cellular therapy. This is the essence of private cord blood storage. In adults, bone marrow stem cells would need to be used as they would not have stored cord blood.

As used herein, the phrase “stem cells” refers both to the earliest renewable cell population responsible for generating cell mass in a tissue or body and the very early progenitor cells, which are somewhat more differentiated, yet are not committed and can readily revert to become a part of the earliest renewable cell population. Methods of ex-vivo culturing stem cells are well known in the art of cell culturing. To this effect, see for example, the text book “Culture of Animal Cells—A Manual of Basic Technique” by Freshney, Wiley-Liss, N.Y. (1994), Third Edition, the teachings of which are hereby incorporated by reference.

The present invention also may include treating one or more lung diseases or conditions by administering a composition of the present invention. In preferred embodiments of the invention, the compositions may be administered intratracheally. In the most preferred embodiments of the invention, intratracheal administration involves contacting or exposing lung tissue, e.g., pulmonary alveoli, to a composition of the present invention.

Some embodiments of the methods and compositions of the present invention may also include one or more surfactants.

Some embodiments of the methods and compositions of the present invention may also include one or more pharmaceutically acceptable carriers. Typical pharmaceutically acceptable carriers include, but are not limited to phosphate buffer solution (PBS), albumin, sterile saline solution, perfluorocarbone, and surfactant solutions.

Some embodiments of the methods and compositions of the present invention the stem cells may be stored prior to administration. In these embodiments of the invention, the stem cell composition may include, but is not limited to DMSO, dextran, autologous plasma, red blood cells, and Pentaspan. The stem cells may also be cultured in fetal calf serum in DMEM, synthetic albumin, or other suitable culture medium, as appropriate for the specific type of stem cell.

The compositions and methods of the present invention may be used to treat or inhibit any lung disease or condition in which it may be desirable to contact one or more lung tissues with a composition comprising stem cells. As used herein, diseases or conditions refers to any disease or condition that results in the pathological alteration of lung function or architecture. Exemplary diseases or conditions include, but are not limited to bronchopulmonary dysplasia (BPD), acute respiratory distress syndrome (ARDS), emphysema, cystic fibrosis (CF), pulmonary hypoplasia, and pulmonary hypertension, and chronic obstructive lung disease (COPD). As used herein the term “inhibiting” refers to slowing, decreasing, delaying, preventing or abolishing.

The compositions and methods of the present invention may be used to treat alveolar damage caused by any disease or condition. Exemplary diseases or conditions include, but are not limited to bronchopulmonary dysplasia (BPD), acute respiratory distress syndrome (ARDS), emphysema, and chronic obstructive lung disease (COPD).

The compositions and methods of the present invention may be used to treat or prevent any oxygen induced lung injury, disease, or condition. Exemplary diseases or conditions include, but are not limited to bronchopulmonary dysplasia (BPD), acute respiratory distress syndrome (ARDS), emphysema, cystic fibrosis (CF), pulmonary hypoplasia, pulmonary hypertension, and chronic obstructive lung disease (COPD).

As noted above, the compositions and methods of the present invention include the use of stem cells. As used herein, stem cells refer to any precursor or progenitor cell that is capable of differentiating into one or more other cell types.

The stem cells may be obtained or isolated from any of a variety of sources, including, but not limited to bone marrow, umbilical cord blood, and/or donor cells from peripheral, circulating blood of any mammal, preferably a human. The invention is directed to purified populations of mammalian stem cells. The stem cells may be endothelial stem cells, muscle stem cells, or neural stem cells. An endothelial stem cell is a stem cell that is capable of maturing into more mature endothelial cells, A muscle stem cell is a stem cell that is capable of maturing at least into a more mature muscle cells. A neural stem cell is a stem cell that is capable of maturing at least into a more mature neural cells.

The stem cells used in the present invention can be of various origin. According to a preferred embodiment of the present invention, the stem and/or progenitor cells are derived from a source selected from the group consisting of hematopoietic cells, umbilical cord blood cells, and mobilized peripheral blood cells. Methods of preparation of stem cells are well known in the art, commonly selecting cells expressing one or more stem cell markers such as CD34, CD133, etc, or lacking markers of differentiated cells. Selection is usually by FACS, or immunomagnetic separation, but can also be by nucleic acid methods such as PCR (see Materials and Experimental Methods hereinbelow). Embryonic stem cells and methods of their retrieval are well known in the art and are described, for example, in Trounson A O (Reprod Fertil Dev (2001) 13: 523), Roach M L (Methods Mel Biol (2002) 185: 1), and Smith A G (Annu Rev Cell Dev Biol (2001) 17:435). Adult stem cells are stem cells, which are derived from tissues of adults and are also well known in the art. Methods of isolating or enriching for adult stem cells are described in, for example, Miraglia, S. et al. (1997) Blood 90: 5013, Uchida, N. et al. (2000) Proc. Natl. Acad. Sci. USA 97: 14720, Simmons, P. J. at al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

The stem cells may be totipotent, pluripotent, multipotent (bipotent or monopotent). Monopotent stem cells are also referred to as progenitor cells. Pluripotent stem cells, bipotent stem cells, and progenitor cells are capable of developing into mature cells either directly or indirectly through one or more intermediate stem or progenitor cells.

Mesenchymal cells are capable of differentiating in a given direction and are capable of expanding. Under normal conditions, mesenchymal cells stay at phase G0, but can shift to phase G1 (initiation of division) when stimulated. Examples of mesenchymal cells include stromal cells and cells having the properties of stromal cells. As used herein, the phrase “mesenchymal cells” refers to cells derived from the mesodermal layer and capable of differentiation. Mesodermal cells can also be characterized, and isolated, by a number of prospective markers: presently, the presence of CD 73 and/or CD105 and/or CD166, CD49b, SH(1), SH(2), SH(3), or SH(4) surface antigens, the absence of CD34+, CD14+, CD45+, and HLA class 1, as well as superior adherence to plastic and multipotent differentiation potential, help to identify cells of mesenchymal lineage from various tissue sources (see Horowitz, Cytotherapy 2000, 2:387-88, and Lee et al, BBRC 2004; 320:273-78, and US Patent Application Nos. 20020058289 and 20040058397 to Thomas, et al).

Examples of cells derived from mesenchymal cells include (1) cells of the cardiovascular system such as endothelial cells or cardiac muscle cells or the precursor cells of the cells of the cardiovascular system, and cells having the properties of these cells; (2) cells of any one of bone, cartilage, tendon and skeletal muscle, the precursor cells of the cells of any one of bone, cartilage, tendon, skeletal muscle and adipose tissue, and the cells having the properties of these cells; (3) neural cells or the precursor cells of neural cells, and the cells having the properties of these cells; (4) endocrine cells or the precursor cells of endocrine cells, and the cells having the properties of these cells; (5) hematopoietic cells or the precursor cells of hematopoietic cells, and the cells having the properties of these cells; and (6) hepatocytes or the precursor cells of hepatocytes, and the cells having the properties of these cells.

Methods of mesenchymal cell culture are well known in the art of cell culturing (see, for example, Friedenstein, at al, Exp Hematol 1976 4, 267-74; Dexter et al. J Cell Physiol 1977, 91:335-44; and Greenberger, Nature 1978 275, 7524), Thus, according to one embodiment of the present invention, mesenchymal cells are derived from a source selected from the group consisting of endothelial cells, cardiac muscle cells, bone cells, cartilage cells, tendon cells, skeletal muscle cells, bone cells, cartilage cells, tendon cells, adipose tissue cells, neural cells, endocrine cells, hematopoietic cells, hematopoietic precursor cells, bone marrow cells, and the precursor cells thereof, hepatocytes, and hepatocyte precursor cells.

In preferred embodiments of the invention, the stem cells may be bone marrow stromal cells, preferably mesenchymal stem cells. For example, in the human experiments described below, the cells are adhered cord blood mononuclear cells processed in medium with fetal calf serum and allowed to adhere to plastic for about four to about seven days. These cells are C34 negative with round and spindled cell morphology, They are autocrine expanded for the duration of their culture. They fit the morphological criteria for MSC-stromal cell like progenitors.

The marrow or isolated mesenchymal stem cells can be autologous, allogeneic or from xenogeneic sources, and can be embryonic or from post-natal sources. Bone marrow cells may be obtained from iliac crest, femora, tibiae, spine, rib or other medullary spaces. Other sources of human mesenchymal stem cells include embryonic yolk sac, placenta, umbilical cord, periosteum, fetal and adolescent skin, and peripheral, circulating blood.

The stem cells of the present invention may be isolated or obtained using any technique, preferably known to those skilled in the art. The inventors have found that the exemplary process described in Example 1 results in a stem cell population suitable for use in a composition of the present invention.

Human mesenchymal stem cells (hMSCs) can be provided as either homogeneous, culture-expanded preparations derived from whole-marrow (or other pre-natal or post-natal source of autologous or allogeneic hMSCs), from hMSC—enriched or heterogeneous cultures containing an effective dose of at least about 10³and preferably at least about 10⁵, preferably about 10⁴ or up to 10⁶, MSCs per milliliter of the composition. The key to effective clinical outcomes, in this embodiment using MSC therapy, is to provide that number of enriched or culture-expanded mesenchymal stem cells to the patient, or about the same number in an optimized medium, which repairs the bone or other tissue defect beyond that in a volume of whole marrow equivalent to that of the defect. This is referred to as the “Regenerative MSC Threshold”, or that concentration of MSCs necessary to achieve direct repair of the tissue defect. The Regenerative MSC Threshold will vary by: 1) type of tissue (i.e., bone, cartilage, ligament, tendon, muscle, marrow stroma, dermis and other connective tissue); 2) size or extent of tissue defect; 3) formulation with pharmaceutical carrier; and 4) age of the patient.

Compositions of the present invention include stem cells as noted above, and may include one or more stem cell metabolites. One skilled in the art recognizes that stem cells release soluble factors that may produce or mediate a therapeutic benefit.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts are Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Gibco/BRL, Nalgene-Nunc International, Sigma Chemical Co., and ICN Biomedicals.

Stem cell types that can be used in vitro or affected in vivo include stem cells that express one or more of CD34+, c-kit, Sca-1, c-mpl, Thy-1, CD38, endoglin, CXCR4, CD41, CD4, IL-6R, IL-3, CD45, Mac-1, AC133, TGF.beta., PECAM, VLA-4, endoglin, CSF-1, LIF, VE-cadherin, steel factor, SSEA-4 and alkaline phosphatase. These markers and methods to obtain and use stem cells have been described, see, e.g., P. J. Quesenberry et al., Stem Cell Reviews, 1;29-36, 2005, S. Sell, Stem Cell Reviews, 1:1-7, 2005, J. S. Van Arnam et al,, Stem Cell Reviews, 1:21-27, 2005, J. R. Bickenbach et al., Stem Cell Reviews, 1:71-77, 2005, C. Dorrell et al., Stem Cell Reviews, 1:61-64, 2005, B. N. Jahagirdar et al., Stem Cell Reviews, 1:53-59, 2005, D. S. Vieyra et al., Stem Cell Reviews, 1:65-69, 2005, A. M. Schor et al., Clin. Orthop. Relat. Res., 313:81-91 1995, M. J. Doherty et al., Crit. Rev. Eukaryot. Gene Expr., 9(1):1-17 1999 and U.S. Pat. Nos. 6,852,533, 6,586,243, 6,528,245, 6,528,245, 6,146,888, 6,174,526, 6,228,640, 6,054,121, 6,004,743, 5,922,847, 6,200,806, 5,843,780, 5,861,315, 5,846,796, 5,840,580, 5,827,742, 5,807,686, 5,804,446, 5,665,557, 5,541,103, 6,897,061, 6,833,269, 6,830,927, 6,680,198, 6,498,018, 6,497,872, 6,060,270, 5,834,312, 5,650,317, 6,872,389, 6,852,330, 6,001,647, 5,981,211, 5,905,041, 6,773,713, 6,767,737, 6,752,831, 6,936,281, 6,866,991, 6,534,084, 6,149,902, 5,733,541, 6,936,281, 6,835,377, 6,737,053, 6,752,834, 6,645,763, 6,576,015, 6,150,164 and 6,699,471 and U.S. patent application publication Nos. 20020173464 and 20050153889.

Culture medium compositions typically include essential amino acids, salts, vitamins, minerals, trace metals, sugars, lipids and nucleosides. Cell culture medium attempts to supply the components necessary to meet the nutritional needs required to grow cells in a controlled, artificial and in vitro environment. Nutrient formulations, pH, and osmolarity vary in accordance with parameters such as cell type, cell density, and the culture system employed. Many cell culture medium formulations are documented in the literature and a number of media are commercially available.

Once the culture medium is incubated with cells, it is known to those skilled in the art as “spent” or “conditioned medium”. Conditioned medium contains many of the original components of the medium, as well as a variety of cellular metabolites and secreted proteins, including, for example, biologically active growth factors, inflammatory mediators and other extracellular proteins.

Preconditioned media ingredients include, but are not limited to those described below. Additionally, the concentration of the ingredients is well known to one of ordinary skill in the art. See, for example, Methods For Preparation Of Media, Supplements and Substrate for Serum-free Animal Cell Cultures. The ingredients include amino-acids (both D and/or L-amino acids) such as glutamine, alanine, arginine, asparagine, cystine, glutamic acid, glycine, histidine, isoleucine, leucine, lysine, methionine, phenylalanine, proline, serine, threonine, tryptophan, tyrosine, and vatine and their derivatives; acid soluble subgroups such as thiamine, ascorbic acid, ferric compounds, ferrous compounds, purines, glutathione and monobasic sodium phosphates.

Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers,

Other ingredients often used in media formulations include fat soluble vitamins (including A, D, E and K) steroids and their derivatives, cholesterol, fatty acids and lipids Tween 80, 2-mercaptoethanol pyramidines as well as a variety of supplements including serum (fetal, horse, calf, etc.), proteins (insulin, transferrin, growth factors, hormones, etc.) antibiotics (gentamicin, penicillin, streptomycin, amphotericin B, etc.) whole egg ultra filtrate, and attachment factors (fibronectins, vitronectins, collagens, laminins, tenascins, etc.).

The media may or may not need to be supplemented with growth factors and other proteins such as attachment factors since many of the cell constructs, particularly the three-dimensional cell and tissue culture constructs described in this application themselves elaborate such growth and attachment factors and other products into the media.

As used herein, soluble factors refers to factors released and present in culture media of stem cells. Examples of soluble factors include but are not limited to vascular endothelial growth factor (VEGF), insulin growth factor (IGF), hepatocyte growth factor (HGF), keratinocyte growth factor (KGF) and other members of the fibroblast growth factor family.

As noted above, the compositions of the present invention may be administered by any suitable route. In preferred embodiments of the invention, the compositions containing stem cells are administered intratracheally. Intratracheal administration refers to the following technique: in our experimental setting intratracheal administration is performed in neonatal rats at 4 days of life (the onset of the alveolar phase of lung development). After halothane anesthesia, the trachea is exposed through a neck-incision. The MSC (25 μl) are then delivered through a tracheal puncture with a short, 30-gauge needle (Becton-Dickinson, Oakville, ON). The incision is then sutured (6.0 prolene, Ethicon, Germany). The rat pups are then allowed to recover. This technique is very well tolerated and allows delivery to the distal airways.

The invention includes compositions comprising one or more pharmaceutically acceptable excipients or carriers. The compositions are used to prepare formulations suitable for human or animal use. Formulations may be designed or intended for oral, rectal, nasal, topical or transmucosal (including buccal, sublingual, ocular, vaginal and rectal) and parenteral (including subcutaneous, intramuscular, intravenous, intradermal, intrathecal, intraocular and epidural) administration. In general, aqueous and non-aqueous liquid or cream formulations are delivered by a parenteral, oral or topical route. In other embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot or a rectal or vaginal suppository. The preferred route may vary with, for example the subject's pathological condition or weight or the subject's response to therapy or that is appropriate to the circumstances. The formulations can also be administered by two or more routes, e.g., subcutaneous injection and buccal or sublingual, where these delivery methods are essentially simultaneous or they may be essentially sequential with little or no temporal overlap in the times at which the compound is administered to the subject.

The formulations include those suitable for the foregoing administration routes. The formulations may conveniently be presented in unit dosage form and may be prepared by any of the methods known in the art of pharmacy. Techniques, dosage forms and excipients such as binders, diluents, disintegrants, viscosity enhancers, stabilizing agents, water absorbing agents, suspending agents and lubricants, are found in, e.g., A. R. Gennaro et al., eds., Remington: The Science and Practice of Pharmacy, Lippincott Williams & Wilkins., Baltimore, Md. 2000, 20^{th} edition; Nema et al., PDA J. Pharm. Sci. Tech. 1997 51;166-171; Pharmaceutical Coating Technology, 1995, G. Cole, et al., editors, Taylor & Francis, ISBN 0 136628915; Pharmaceutical Dosage Forms, 1992 2.sup.nd revised edition, volumes 1 and 2, H. A. Lieberman, et al., editors, Marcel Dekker, ISBN 0824793870; Pharmaceutical Preformulation, 1998, pages 1-306, J. T. Carstensen, Technomic Publishing Co. ISBN 1566766907; Encyclopedia of Pharmaceutical Technology, volumes 1, 2 and 3, 2.sup.nd edition, 2002, J. Swarbrick and J. C Boylan, editors, Marcel Dekker, Inc., New York, N.Y.; and A. H. Kibbe, ed. Handbook of Pharmaceutical Excipients, 3.sup.rd ed., 2000, American Pharmaceutical Association, Washington, D.C.

As noted above, the compositions of the present invention may include one or more surfactants, or may be used in combination with one or more surfactant therapies. Surfactant, as used herein, refers to any surface active agent, including but not limited to wetting agents, surface tension depressants, detergents, dispersing agents, emulsifiers. Particularly preferred are those that from a monomolecular layer over pulmonary alveolar surfaces, including but not limited to lipoproteins, lecithins, and sphygomyelins.

Exemplary surfactants include, but are not limited to surfactant protein A, surfactant protein B, surfactant protein C, surfactant protein D, and mixtures and combinations thereof. Commercially available surfactants include but are not limited to Survanta, bLES, Infasurf, Curosurf, HL-10, Alveofact, Surfaxin, Venticute, ALEC, and Exosu rf.

The compositions and methods of the present invention engraft in the injured lung. Engraftment may be assessed by counting under a confocal microscope the number of green cells (MSC labeled with a green fluorescent marker, e.g., CFSE, prior to injection) that have nuclei (stained blue) per high power lung field examined.

The compositions and methods of the present invention adopt a lung phenotype. By counting the number of cells that are green (MSC) and red (stained forsurfactant protein C, a marker of alveolar type 2 cells), we can also determine how many cells engrafted and adopted an alveolar type 2 cell phenotype in vivo. In vitro, bone-marrow derived MSC co-cultured with lung tissue for 6 days express surfactant protein C as assessed by immunofluorescence and quantitative real time RT-PCR. Cell differentiation of various lineages is a well-documented process and requires no further description herein. As used herein the term differentiation is distinct from maturation which is a process, although some times associated with cell division, in which a specific cell type matures to function and then dies, e.g., via programmed cell death.

Homogeneous human mesenchymal stem cell compositions are obtained by culturing adherent marrow or periosteal cells; the mesenchymal stem cells may be identified by specific cell surface markers which are identified with unique monoclonal antibodies. A method for obtaining a cell population enriched in mesenchymal stem cells is described, for example, in U.S. Pat. No. 5,486,359.

The present invention can be used for gene therapy. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

According to the methods of the present invention, the ex-vivo expanded cells are implanted into a subject in need of thereof. In one embodiment, the subject is a human. As used herein, the phrase “in need thereof” indicates the state of the subject, wherein enhancement of one or more organs or tissues is desirable. In any of the methods of this aspect of the present invention, the donor and the recipient of the stem and/or progenitor cells can be a single individual or different individuals, for example, allogeneic or xenogeneic individuals. When allogeneic transplantation is practiced, regimes for reducing implant rejection and/or graft vs. host disease, as well know in the art, should be undertaken. Such regimes are currently practiced in human therapy. Most advanced regimes are disclosed in publications by Slavin S. et al., e.g., J Clin Immunol (2002) 22: 64, and J. Hematother Stem Cell Res (2002) 11: 265), Gur H. et al. (Blood (2002) 99: 4174), and Martell M F et al, (Semin Hematol (2002) 39: 48), which are incorporated herein by reference.

The compositions and methods of the present invention improve survival, as shown in the examples.

These preliminary data highlight the therapeutic potential of stem cells for lung diseases that currently lack efficient treatments, Furthermore, the source of stem cells is readily available, easily accessible and does not encounter the same ethical issues than other stem cell sources

Other therapeutic airway delivery techniques to consider include nebulization in intubated and non-intubated patients or aerosol delivery, e.g., with a “puffer” (routinely used by asthmatic patients).

The compositions of the present invention can be used to treat, ameliorate, prevent or slow the progression of a number of pulmonary conditions or their symptoms such as 1, 2, 3 or more of cystic fibrosis, bronchiectasis, car pulmonale, pneumonia, lung abcess, acute bronchitis, chronic bronchitis, a chronic obstructive pulmonary disease (COPD) condition, bronchopulmonary dysplasia, emphysema, pneumonitis, e.g., hypersensitivity pneumonitis or pneumonitis associated with radiation exposure, alveolar lung diseases and interstitial lung diseases, e.g., associated with asbestos, fumes or gas exposure, aspiration pneumonia, pulmonary hemorrhage syndromes, amyloidosis, connective tissue diseases, systemic sclerosis, ankylosing spondylitis, allergic granulomatosis, granulomatous vasculitides, asthma, e.g., mild intermittent asthma, mild persistent asthma, moderate persistent asthma, severe persistent asthma, acute asthma, chronic asthma, atopic asthma, allergic asthma or idiosyncratic asthma, cystic fibrosis and associated conditions, e.g., allergic bronchopulmonary aspergillosis, chronic sinusitis, pancreatic insufficiency, lung or vascular inflammation, bacterial or viral infection, e.g., Haemophilus influenzae, S. aureus, Pseudomonas aeruginosa or RSV infection or an acute or chronic adult or pediatric respiratory distress syndrome (RDS) suh as grade I, II, III or IV RDS or an RDS associated with, e.g., sepsis, pneumonia, reperfusion, atelectasis or chest trauma. Chronic obstructive pulmonary diseases include conditions where airflow obstruction is located at upper airways, intermediate-sized airways, bronchioles or parenchyma, which can be manifested as, or associated with, tracheal stenosis, tracheal right ventricular hypertrophy pulmonary hypertension, polychondritis, bronchiectasis, bronchiolitis, e.g., idiopathic bronchiolitis, ciliary dyskinesia, asthma, emphysema, connective tissue disease, bronchiolitis of chronic bronchitis or lung transplantation. The composition can be used to treat or ameliorate acute or chronic asthma or their symptoms or complications, including airway smooth muscle spasm or hyperresponsiveness, airway mucosa edema, increased mucus secretion, excessive, T cell activation, airway epithelium injury or desquamation, atelectasis, cor pulmonale, pneumothorax, subcutaneous emphysema, dyspnea, coughing, wheezing, shortness of breath, tachypnea, fatigue, decreased forced expiratory volume in the 1^{st} second (FEV.₁), arterial hypoxemia, respiratory acidosis, inflammation including unwanted elevated levels of mediators such as IL-4, IL-5, IgE, histamine, substance P, neurokinin A, calcitonin gene-related peptide or arachidonic acid metabolites such as thromboxane or leukotrienes (LTD.₄ or LTC,₄), and cellular airway wall cellular infiltration, e.g., by eosinophils, lymphocytes, macrophages or granulocytes.

Any of these and other pulmonary conditions or symptoms are described elsewhere, e.g., The Merck Manual, 17^{th} edition, M. H. Beers and R. Berkow editors, 1999, Merck Research Laboratories, Whitehouse Station, N.J., ISBN 0911910-10-7, or in other references cited herein, In some of these conditions where inflammation plays a role in the pathology of the condition, the compositions can ameliorate or slow the progression of the condition by reducing damage from inflammation. In other cases, the compositions act to limit pathogen replication or pathogen-associated lung tissue damage. Other standard treatments can be combined with the use of the compositions to treat these conditions or symptoms, e.g., asthma, RDS or COPD, including the use of anticholinergic agents, β2-adrenoreceptor agonists such as formoterol or salmeterol, corticosteroids, antibiotics or antihypertension agents.

Other combinatorial therapeutic strategies include gene therapy, e.g., by over-expressing a given gene of interest in stem cells, before administration.

The use of a composition of the present invention to treat, ameliorate or slow the progression of conditions such as CF can be optionally combined with other suitable treatments. For CF, this includes, e.g., one, two or more of oral or aerosol corticosteroid treatment, ibuprofen treatment, DNAse or IL-10 treatment, diet control, e.g., vitamin E supplementation, vaccination against pathogens, e.g., Haemophilus influenzae, or chest physical therapy, e.g., chest drainage or percussion.

Humans or other subjects who have one or more of these conditions can be treated with other suitable therapeutics. Pulmonary conditions that can be treated with the compositions and other therapeutic methods and agents that can be used in conjunction with the compositions have been described in detail, see, e.g., Harrison's Principles of Internal Medicine, 15^{th} edition, 2001, E. Braunwald, et al., editors, McGraw-Hill, New York, N.Y., ISBN 0-07-007272-8, especially chapters 252-265 at pages 1456-1526; Physicians Desk Reference 54^{th} edition, 2000, pages 303-3251, ISBN 1-56363-330-2, Medical Economics Co., Inc., Montvale, N.J. Treatment of any of these respiratory and pulmonary conditions using a composition may be accomplished using the treatment regimens described herein. For chronic conditions, intermittent dosing can be used to reduce the frequency of treatment. Intermittent dosing protocols are as described herein.

In a preferred embodiment, the method further comprises administering at least one bioactive factor that further induces or accelerates the differentiation of such mesenchymal stem cells into the osteogenic lineage. Preferably, the cells are contacted with the bioactive factor ex vivo, while in the matrix, or injected into the defect site at or following the implantation of the composition of the invention. It is particularly preferred that the bioactive factor is a member of the TGF-β superfamily comprising various tissue growth factors, particularly bone morphogenic proteins, such as at least one selected from the group consisting of BMP-2, BMP-3, BMP-4, BMP-6 and BMP-7.

As noted above, mesenchymal cells may be cultured. Two bone-marrow culture systems introduced in the mid-1970's have evolved as favored media for the in vitro analysis of mesengenesis and hematopoiesis. The Friedenstein culture system is based on the isolation of non-hematopoietic cells through their tendency to adhere to plastic. Once isolated, a monolayer of homogeneous, undifferentiated stromal cells is then grown in the culture medium, in the absence of hematopoietic cells. The stromal cells from this system have the potential to differentiate into discrete mesenchymal tissues, namely bone, cartilage, adipose tissue and/or muscle depending on specific growth supplements (Friedenstein, et al, Exp Hematol 1976 4, 267-74).

In 1977, Dexter, et al. developed another bone marrow culture system for the study of hematopoiesis. (Dexter et al. J Cell Physiol 1977, 91:335-44). The Dexter culture does not require isolation of the mesenchymal cells before culturing, thus the monolayer of stromal cells is grown in the presence of hematopoietic cells. Greenberger later modified the Dexter system by the addition of hydrocortisone to the culture medium, making it more reproducible (Greenberger, Nature 1978 275, 752-4).

Thiede et al (U.S. Pat. No. 6,030,836) disclose the use of co-cultured bone marrow hematopoietic stem cells and mesenchymal stem cells (or adipocytes), enriching the CD34+, CD34+/90+ and CD34+/CD14+ fraction of hematopoietic stem cells for engraftment. McIntosh et al (U.S. Pat. Nos. 6,368,636 and 6,875,430) teach the reduction of immune response to cellular transplant, and reduction in graft versus host disease, by infusion of mesenchymal stem cells before, after, or along with the transplanted cells. Seshi, et al (US Patent Application No. 2003000308) teaches the isolation of mesenchymal progenitor cells expressing different multiple cellular differentiation markers, such as fat, osteoblasts, smooth muscle and fibroblast markers, for treatment of graft versus host disease, and for enhanced transplantation.

In a preferred embodiment, the source of the stem cell population is an unfractionated mononuclear cell preparation, not having been enriched for CD34+ or other hematopoietic stem cells. In another embodiment, the stem cells are identified by stem cell markers such as CD34+, CD34+/CD38−, CD133+, CD34+/Lin−, and other stem cell markers known in the art. In yet another embodiment, the source of the stem cell population for co-culture with mesenchymal cells is stem cells having been enriched for hematopoietic stem cells by selection according to stem cell markers. In yet a further embodiment, the source of the stem cell population for co-culture with mesenchymal cells is an expanded, undifferentiated stem cell population, following short- or long-term expansion in accordance with known procedures.

It will be appreciated, in the context of the present invention, that the cell culture can be a two dimensional cell culture system, or a three dimensional cell culture system. Two-dimensional cell culture systems are defined as cultures in which cells are grown as confluent monolayers which make the surface suitable for attachment of other cells, such as feeder layers. For example, the growth of glioma on confluent feeder layers of normal fetal intestine has been reported. While the growth of cells in two dimensions is a convenient method for preparing, observing and studying cells in culture, allowing a high rate of cell proliferation, it lacks the cell-cell and cell-matrix interactions characteristic of whole tissue in vivo. Investigators have explored the use of three-dimensional substrates such as collagen gel (Douglas et al., 1980, In Vitro 16:306-312; Yang et al., 1979, Proc. Natl. Acad. Sci. 76:3401; Yang et at, 1980, Proc. Natl. Acad. Sci. 77:2088-2092; Yang et al., 1981, Cancer Res. 41:1021-1027); cellulose sponge, alone (Leighton et al., 1951, J. Natl. Cancer Inst. 12:545-561) or collagen coated (Leighton et al., 1968, Cancer Res. 28:286-296); a gelatin sponge, Gelfoam (Sorour et at, 1975, J. Neurosurg. 43:742-749). Three dimensional cell culture systems are systems which allow the cells to grow in multiple layers, thus creating a three-dimensional cell culture system which may overcome the limitations of contact inhibition found in two-dimensional systems. Three dimensional culture systems are usually characterized by provision of a support or matrix for growth of cells, as detailed hereinabove. Many cell types and tissues can be grown in the three-dimensional culture system. Methods for growth of cells in such three dimensional cultures are described in detail in the art, for example, U.S. Pat. Nos. 5,160,490, 5,032,508, 5,785,964 and 5,858,721, all to Naughton, et al.

Peled et at (IL 2004/00064) demonstrated that unselected cells from the total nucleated cell (TNC) fraction of cord blood can be expanded ex-vivo while inhibiting differentiation thereof, and used for transplantation. Thus, in one embodiment of the present invention, the population of cells comprising stem cells is unselected mononuclear cells. PCT IL03/00681 to Peled, et al, which is incorporated by reference as if fully set for herein, discloses the use of molecules such as copper chelators, copper chelates and retinoic acid receptor (RAR) antagonists which are capable of repressing differentiation and stimulating and prolonging proliferation of hematopoietic stem cells when the source of cells includes the entire fraction of mononuclear blood cells, namely non-enriched stem cells.

As described hereinabove, prior to implantation the stem and/or progenitor cells can be co-cultured with mesenchymal cells ex-vivo under conditions allowing for cell proliferation and, at the same time, substantially inhibiting differentiation thereof. According to preferred embodiments of the present invention, providing the stem cells with the conditions for ex-vivo cell proliferation comprises providing the cells with nutrients and with cytokines. Preferably, the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-.alpha. and thrombopoietin. It will be appreciated in this respect that novel cytokines are continuously discovered, some of which may find uses in the methods of cell expansion of the present invention.

The terms “effective amount”, “effective dose” or the like with reference to the compositions of the present invention mean an amount of the composition that is sufficient to elicit a desired or detectable response, e.g., detectable restoration of normal immune responsiveness in an immunodeficient subject to which it is administered, e.g., a human, or to detectable modulation or amelioration of cellular parameter or a clinical condition or symptom or a detectable amount for analytical or other characterization use.

Terms such as “use”, “treat”, “treatment”, “address” or the like in the context of using the treatment methods or other methods disclosed herein mean that a composition is administered to a subject, delivered to the subject's tissues or contacted with tissues, cells or cell free systems in vivo or in vitro, e.g., as described herein or a reference cited herein, Typically such use or treatment results in, e.g., (1) detectable improvement in or amelioration of the condition or symptom being treated, (2) detectable modulation in the activity, level or numbers of a relevant biomolecule, therapeutic immune cell population or a pathological cell population, (3) slowing of the progression of a condition or delaying its onset, or reduction of the severity of a symptom(s) of the condition or (4) another detectable response as described herein. Any such amelioration may be transient, e.g., lasting for at least a few, e.g., about 1, 2 or 4 hours to about 10, 12 or 24 hours or lasting for days, e.g., about 1, 2, 3 or 4 days to about 5, 7,10 or more days. Amelioration may be prolonged, e.g., lasting from about 10, 12, or 14 days, to about 18, 21, 28, 35, 42, 49, 60 or more days, or amelioration may be permanent. A treatment may slow the progression of a disease or symptom or it may reduce the severity thereof, e.g., onset of a disease or a symptom may be delayed in at least some subjects for about 1-24 hours, about 2-10 days, about 2-30 days or for about 1-5 years compared to subjects who are not treated with sufficient amounts of the composition.

Such tissue culture systems will normally contain growth media and growth factors, substrates and differentiation modulators that are often used to expand or maintain stem cells in vitro. Growth factors, substrates and differentiation modulators that can be used include one or more of thrombopoietin, erythropoietin, IL-2, IL-3, IL-6, G-CSF, GM-CSF, stem cell factor, FLT-3 ligand, a fibroblast growth factor such as FGF-4 or basic fibroblast growth factor, TGF.alpha., TGF-.beta.1, insulin, oncostatin-M, collagen, laminin, vascular endothelial growth factor and 5-azacytidine.

Toxicity and therapeutic efficacy of the drugs can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

### VI. EXAMPLES

### Example 1

a. We tested the hypothesis that bone-marrow (BM)-derived and human umbilical cord blood (hUCB)-derived mesenchymal stem cells (MSC) would be able to prevent oxygen-induced lung injury in a newborn rat model mimicking human BPD. Because the lung is relatively accessible for drug delivery via the airway and because this route of administration is clinically pertinent, we choose to administer the stem cells via intratracheal injection. The animal model involves exposure of newborn rat pups to 95% oxygen from birth to 14 days postnatal. This creates lung injury similar to human BPD and is irreversible in this animal model, suggesting that the intrinsic repair mechanisms of the lung are overwhelmed. In vivo intra-tracheal administration of mesenchymal stem cells is performed through intratracheal puncture at 4 days of age.

After halothane anesthesia, the trachea is exposed through a neck-incision. Stem cells (25 μl) are then delivered through a tracheal puncture with a short, 30-gauge needle (Becton-Dickinson, Oakville, Ontario, Canada). The incision is then sutured (6.0 Prolene, Ethicon, Germany). The rat pups are then allowed to recover. This technique is very well tolerated and allows efficient delivery to the distal airways.

A strength of this approach is that it mimics the clinical setting and could theoretically be used (if safe and effective) to treat intubated patient with respiratory distress in the intensive care unit, such as premature infants who are likely to develop BPD or to treat infants and adults with Acute Respiratory Distress Syndrome (ARDS). We have now combined our stem cell injection with surfactant. Surfactant is routinely administered to premature infants with hyaline membrane disease (surfactant deficiency)-the same population that is at risk for BPD, and to adults with ARDS.

### Example 2

Our results show that BM-derived MSC and hUCB-derived stem cells engraft into the alveolar epithelium and prevent alveolar damage. To explore the mechanisms of this therapeutic benefit, we performed in vitro (cell culture experiments) and showed that BM-derived MSC, when in contact with oxygen-injured lung can adopt a lung phenotype by expressing surfactant protein C (SP-C) a specific marker for alveolar type 2 cells.

### Example 3

Isolation of Murine MSCs and Alveolar Type II Cells. MSCs were isolated from mouse bone marrow as described (7) except that whole bone marrow was plated at a density of 1.46×10₆ cells per cm2 and cultured for 8-10 days before harvest. MSCs (up to 40×10₆ cells) were added to M-280 Dynabeads (five beads per cell; Dynal, Oslo) conjugated to an anti-CD11b antibody (10 μg per mg of beads; PharMingen) in a volume of 1 ml and incubated on a rotator at 4° C. for 45 min. Successive rounds of immunodepletion by using antibodies against CD34 and CD45 (PharMingen) were conducted similarly. Immunodepleted cells were suspended in Hanks' balanced salt solution and used for further experiments as described. Alveolar epithelial type II cells were isolated from the lung tissue, Approximately 2×10₆ cells were used to prepare genomic DNA for real-time PCR. Alternatively, 5×10₄ cells were cultured and fixed on positively charged slides for analysis by fluorescence in situ hybridization (FISH).

### Example 4

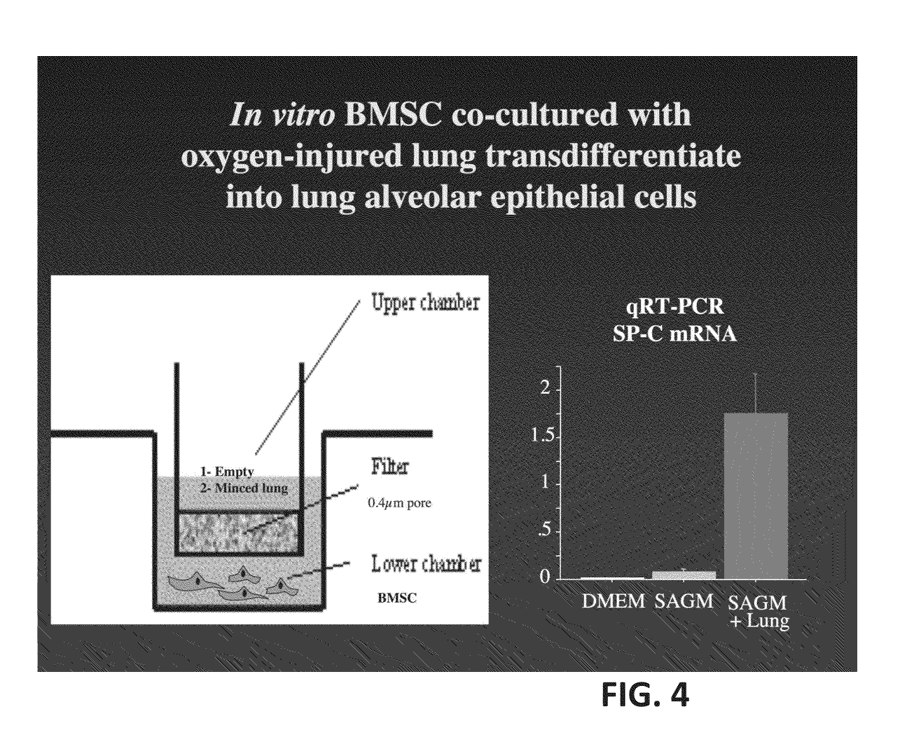

Bone marrow stem cells were co-cultured In vitro with oxygen-injured lung cells, and transdifferentiated into lung alveolar epithelial cells. The results are shown in FIG. 4.

### Example 5

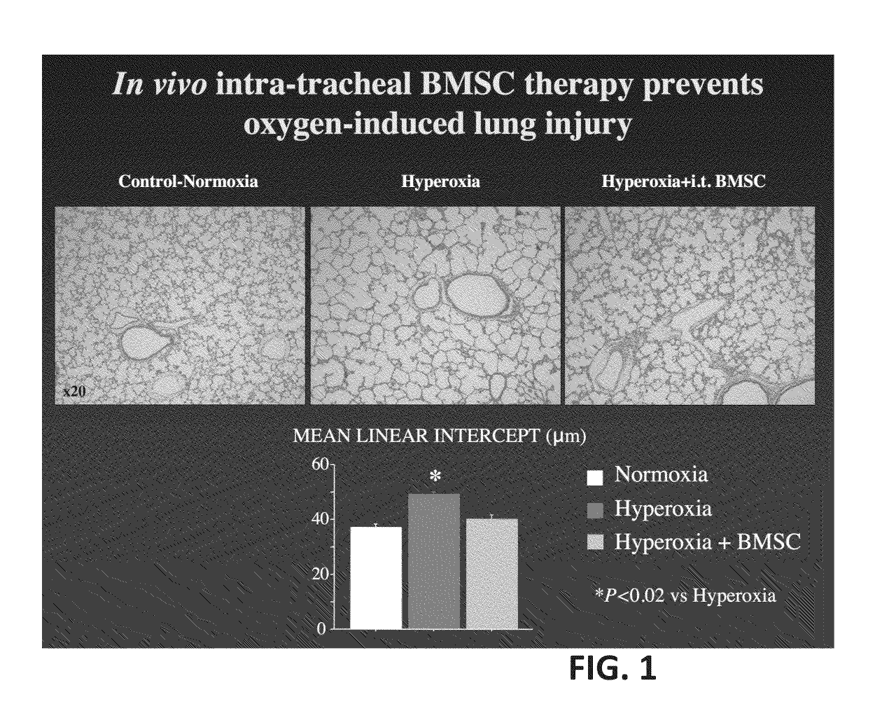

Bone marrow stem cells were administered intratracheally, and were shown to prevent oxygen-induced lung injury. The results are shown in FIG. 1.

### Example 6

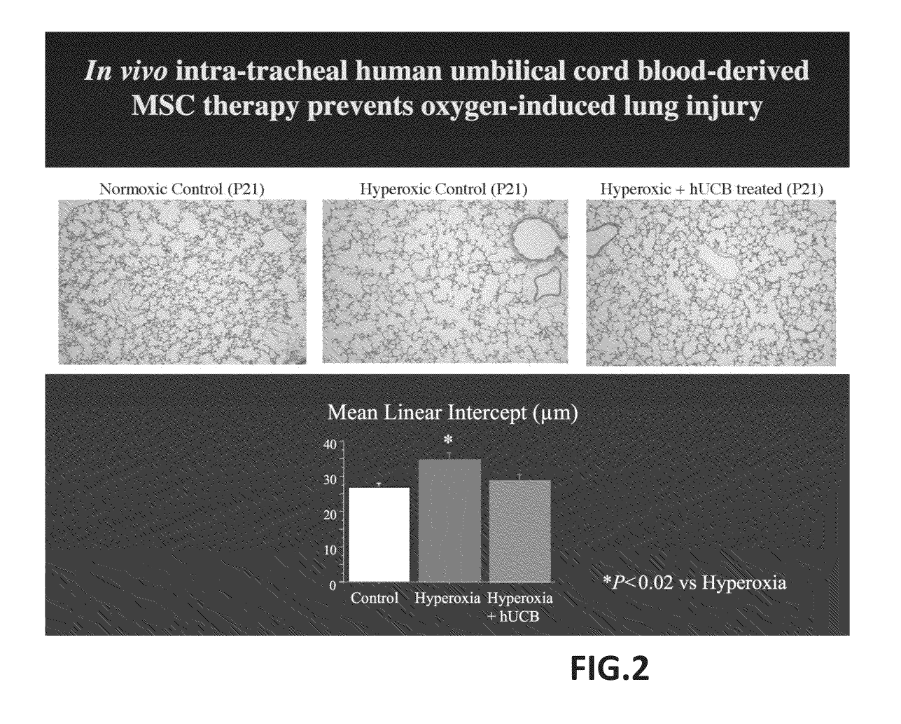

Mesenchymal stem cells were administered intratracheally, and were shown to prevent oxygen-induced lung injury. The results are shown in FIG. 2.

### Example 7

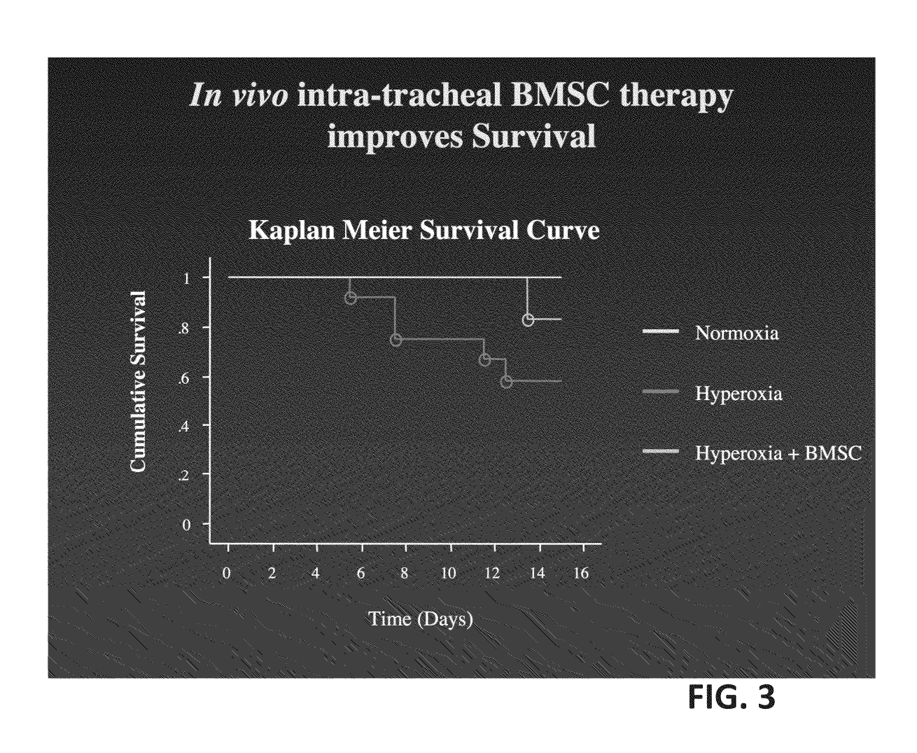

Bone marrow stem cells were administered in vivo intratracheally and were shown to increase survival rates, The results are shown in FIG. 3.

### Example 8

### Techniques of Isolating Stem Cells

A mixture of cells from a suitable source of endothelial, muscle, and/or neural stem cells, as described above, is harvested from a mammalian donor by methods known in the art. A suitable source is the hematopoietic microenvironment.

For example, circulating peripheral blood, preferably mobilized (i.e., recruited) as described below, may be removed from a patient. Alternatively, bone marrow may be obtained from a mammal, such as a human patient, undergoing an autologous transplant.

The mixture of cells obtained is exposed to a molecule that binds specifically to the antigen marker characteristic of stem cells. The binding molecule is preferably an antibody or a fragment of an antibody. A convenient antigen marker is a VEGF receptor, more specifically a FLK-1 receptor.

The cells that express the antigen marker bind to the binding molecule. The binding molecule distinguishes the bound cells from unbound cells, permitting separation and isolation. If the bound cells do not internalize the molecule, the molecule may be separated from the cell by methods known in the art. For example, antibodies may be separated from cells by a short exposure to a solution having a low pH, or with a protease such as chymotrypsin.

The molecule used for isolating the purified populations of stem cells is advantageously conjugated with labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS), and the like.

Any technique may be used for isolation as long as the technique does not unduly harm the stem cells. Many such methods are known in the art.

In one embodiment, the binding molecule is attached to a solid support. Some suitable solid supports include nitrocellulose, agarose beads, polystyrene beads, hollow fiber membranes, magnetic beads, and plastic petri dishes. For example, the binding molecule can be covalently linked to Pharmacia Sepharose 6 MB macro beads. The exact conditions and duration of incubation for the solid phase-linked binding molecules with the crude cell mixture will depend upon several factors specific to the system employed, as is well known in the art.

Cells that are bound to the binding molecule are removed from the cell suspension by physically separating the solid support from the remaining cell suspension. For example, the unbound cells may be eluted or washed away with physiologic buffer after allowing sufficient time for the solid support to bind the stem cells.

The bound cells are separated from the solid phase by any appropriate method, depending mainly upon the nature of the solid phase and the binding molecule. For example, bound cells can be eluted from a plastic Petri dish by vigorous agitation. Alternatively, bound cells can be eluted by enzymatically “nicking” or digesting an enzyme-sensitive “spacer” sequence between the solid phase and an antibody, Suitable spacer sequences bound to agarose beads are commercially available from, for example, Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

Methods for removing unwanted cells by negative selection are also known. For example, unwanted cells in a starting cell population are labeled by an antibody, or by a cocktail of antibodies, to a cell surface protein characteristic of Lin+ cells. The unwanted antibody-labeled cells are removed by methods known in the art, For example, the labeled cells can be immobilized on a column that binds to the antibodies and captures the cells.

Alternatively, the antibody that binds the cell surface proteins can be linked to magnetic colloids for capture of unwanted cells on a column surrounded by a magnetic field. This system is currently available through StemCell Technologies Inc., Vancouver, British Columbia, Canada, The remaining cells that flow through the column for collection are enriched in cells that do not express the cell surface proteins that the tetrameric antibodies were directed against. The antibody cocktail that can be used to deplete unwanted Lin+ cells can be custom made to include antibodies against lineage specific markers, such as, for example, CD2, CD3, CD4, CD5, CD8, CD10, CD11b, CD13, CD14, CD15, CD16, CD19, CD20, CD24, CD25, CD28, CD29, CD33, CD36, CD38, CD41, CD56, CD66b, CD66e, CD69, and glycophorin A. The desired cells that lack these markers are not lineage committed, i.e. Lin−.

### Example 9

### Animal Model

All procedures and protocols were approved by the Animal Health Care Committee of the University of Alberta. Experimental BPD was induced as previously described. Sprague-Dawley (Charles River) rat pups were exposed to normoxia (21% oxygen, control group) or hyperoxia (95% oxygen, BPD model) from birth to postnatal day 14 (P14) in sealed plexiglass chambers with continuous oxygen monitoring (BioSpherix, Redfield, N.Y.). Litters were normalized so all cages had an equal number of pups at PO to control for the effects of litter size and nutrition on growth. Dams were switched between normoxic and hyperoxic chambers every 48 hours to prevent damage to the dam's lungs. Rat pups were sacrificed at P21 via an intraperitoneal injection of pentobarbital.

### Mesenchymal Stem Cell Harvest and Cell Culture

Bone marrow was harvested from adult Sprague-Dawley rats by excising the femur and tibia and flushing the marrow with Dulbecco's Modified Eagle Medium (DMEM; Gibco Canada, Burlington, Ontario, Canada). The isolated marrow was dissociated with a 21 gauge needle and plated into a tissue culture flask. After overnight adherence, the media was aspirated and adhered cells were rinsed with PBS, and the media replenished. Adhered cells were allowed to grow to ˜75% confluency, then trypsinized and reseeded at a density of 10⁵ cells/cm². This procedure was performed for 2 passages. Pulmonary artery smooth muscle cells, used as control cells, were obtained from 20 week old Sprague-Dawley.

### Fluorescence-Activated Cell Sorting of Cell Surface Markers

Phycoerythrin labeling for rat monoclonal antibodies against CD31 (PECAM-1), CD34, CD44 (H-CAM), CD45 (leukocyte common antigen (LCA)), CD54 (ICAM-1), CD73 SH4) and CD90 (Thy-1) (Santa Cruz, Santa Cruz, Calif., USA) were utilized according to the manufacturer's protocol. These antibodies were selected in accordance with the position statement for the minimal criteria to define a mesenchymal stem cell from the International Society for Cellular Therapy. Irrelevant isotype-identical antibody, immunoglobulin-G (IgG), served as negative control for auto-fluorescence. Specific and unspecific antibody binding was detected with a secondary phycoerythrin-labeled anti-mouse antibody. Samples were analyzed by collecting 10,000 cellular events. Cells were analyzed using a FACScan (Becton Dickinson, Franklin Lakes, N.J., USA) and CellQuest Software.

### Stem Cell Lineage Differentiation Assay

The lineage differentiation assay was performed in accordance with the position statement for the minimal criteria to define a mesenchymal stem cell from the International Society for Cellular Therapy. Passage to bone marrow-derived MSC differentiation potential was evaluated as follows: Adipogenic induction. Cells were cultured for 14 days in a-MEM (minimal essential media) containing 10% (v/v) FCS (fetal calf serum), 100 μM isobutyl methylxanthine, 60 μM indomethacin, 1 μg/ml insulin and 0.5 μM hydrocortisone (Sigma Canada, Oakville, ON, Canada). Media was changed every 3 days. Adipogenic differentiation was shown by cellular accumulation of large-sized (≈5 μm in diameter) lipid vacuoles that were stained with Oil-red O and counterstained with DAPI. Osteogenic induction. Cells were cultured for 21 days in α-MEM containing 20% (v/v) FCS, 0.1 μM dexamethasone, 2 mM β-glycerophosphate and 150 μM ascorbic acid; medium was changed every 3 days. Mineralization areas were revealed by Alizarin red staining (Sigma Canada, Oakville, ON, Canada). Chondrogenic induction. Cells at 80% confluency were trypsinized with 0.05% (v/v) trypsin-EDTA and resuspended in low-glucose DMEM containing 1 mM dexamethasone, 1 mM sodium pyruvate, 1× insulin-transferrin-selenium, 17 mM ascorbic acid, 35 mM praline and 10 ng/ml transforming growth factor β1. Viable cells were counted and seeded at a density of 5×10⁵ cells per pellet in 15-cm³ conical tubes. Cells were gently centrifuged to the bottom of the tubes and allowed to form compact cell pellets, then incubated in a humidified atmosphere at 37° C. with 5% CO₂ with medium changes every 3 days. After 21 days in culture, pellets were embedded in paraffin. Cartilage glycosaminoglycans were detected by staining with Safranin O.

### RT-PCR Analysis for Lineage Conversion

Total RNA was extracted from undifferentiated (control) and differentiated BM-derived MSCs and analyzed by RT-PCR with primers specific for rat acidic ribosomal phosphoprotein P0 (Rplp0), lipoprotein lipase (Lpl), peroxisome proliferator activated receptor gamma 2 (Pparg2), bone gamma-carboxyglutamate protein (Bgiap), runt-related transcription factor 2 (Runx2), pro-α1(II) collagen (Col2a1) and pro-α1(X) collagen (Coll10a1). Amplified cDNA fragments were electrophoresed through a 2% (w/v) agarose gel, stained by ethidium bromide, and photographed under an ultraviolet light transilluminator,

### In Vivo Experimental Design

Newborn rat pups were randomized to four groups: (1) normoxia (21% oxygen, control group); (2) hyperoxia (95% oxygen, BPD group); (3) hyperoxia+MSCs (BPD treatment group); and (4) hyperoxia+pulmonary artery smooth muscle cells (PASMCs; BPD treatment control group). MSCs or PASMCs were administered at P4 via an IT injection (1.0×10⁵/cells per animal). Before administration, MSCs and PASMCs were labeled with the intra-vital green fluorescent dye 5(6)-Carboxyfluorescein diacetate N-succinimidyl ester (CFSE) (Sigma Canada, Oakville, ON, Canada) according to the manufacturer's protocol. Oxygen exposure was ceased at P14, and all tissues were harvested at P21.

### Lung Morphometry

Lungs were inflated and fixed via the trachea with a 4% formaldehyde solution at a constant pressure of 20 cm H₂O. After tracheal ligation, the lungs were placed in fixative overnight. Lung volume was measured via water displacement. Lungs were processed and paraffin embedded. Four ₄m thick serial sections were taken along the longitudinal axis of the right middle lobe. The fixed distance between the sections was calculated to allow systematic sampling of 10 sections across the whole lobe. Lung sections were stained with hematoxylin and eosin. Alveolar development was quantified using the mean linear intercept method. This method quantifies the average distance between adjacent structures for blood-gas exchange,

### MSC Engraftment

Lungs from P14 and P21 animals were inflated with Tissue-Tek Optimal Cutting Temperature (OCT) Media (Ted Pella, Inc. Redding, Calif.) and subsequently frozen in a block of OCT. Lungs were stained for surfactant protein-C (SP-C) and cellular nuclei (DAPI). Lung slices were imaged using a confocal microscope. Five longitudinal slices were used per animal and five random fields per slide were imaged. Total cellular number was based on DAPI staining, type II AECs were identified by the staining of SP-C, MSCs by the presence of green fluorescence (CFSE), and “transdifferentiated” MSCs were identified by the co-localization of SP-C with CFSE.

### Barium Angiogram

Barium was instilled into the pulmonary vasculature. The animals were anesthetized with a dose of pentobarbital. Once in surgical plane, with the heart still beating, the chest was opened and 5 μl of heparin was injected directly into the right ventricle. The pulmonary artery was cannulated with a 22 gauge cannula. The lungs were flushed with a 10% heparin 90% saline solution (1 ml). Barium sulfate (Polibar) was infused into the lungs via the pulmonary artery catheter until it flowed out of the left atrium. The barium was imaged with a rodent SPECT-CT (FLEX Pre-clinical platform) and the Amira software package (Gamma Medica, Northridge, Calif., USA).

### Mercox Vascular Casting and Scanning Electron Microscopy

Mercox catalyst and resin (Ladd Research Industries), at a 50:1 ratio, was infused into the pulmonary vasculature in the same manner as the barium angiogram. The soft tissue was dissolved by placing the lungs in 20% KOH for two days. The resulting vascular casts were rinsed with distilled water, air dried at 400 for one hour, and mounted on a stub. The casts were sputter-coated with gold (Edwards S150B; Edwards) and imaged with a Hitachi SEM S-2500.

### Right Ventricular Hypertrophy

Right ventricular hypertrophy was assessed post-mortem. The atria and great vessels of the heart were dissected away. The right ventricle free wall was separated from the left ventricle and the septal wall. The tissue was allowed to dry overnight, and was weighed the following day.

### Pulmonary Artery Acceleration Time

Pulmonary artery acceleration time, a valid measure of mean pulmonary arterial pressure in rodents was assessed with Doppler echocardiography.

In vitro Co-Culture Assay

MSCs were seeded in the bottom of a 24-well culture plate in either DMEM (Gibco) or small airway growth media (SAGM, Clonetics). We exposed the MSCs to normal growth media (DMEM), small airway growth media (SAGM), SAGM+hyperoxic damaged lung tissue, DMEM+normoxic lung tissue, DMEM+hyperoxic damaged lung tissue, and DMEM+liver tissue via a modified Boyden chamber (Corning Inc, Corning, N.Y.) with a 0.4 μm mesh separating the upper and lower chambers. The cells were stained for surfactant protein-C (SP-C) and cellular nuclei (DAPI). The cells were imaged with a confocal microscope. Total RNA from cells was obtained using an RNEasy mini kit (Qiagen). Primer for SP-C was obtained from Applied Biosystems. The TaqMan One-Step RT-PCR Master Mix reagent kit (Applied Biosystems, Foster City, Calif., USA) was used to quantify the copy number of cDNA targets. The reaction used 50 ng RNA in 50 μl using relevant (SP-C) primer (500 nM), and TaqMan probe (200 nM). The assay was performed using ABI PRISM 7700 Sequence Detector System (Applied Biosystems, Foster City, Calif., USA). Reverse transcription proceeded for 30 min at 48° C. AmpliTaq Gold activation occurred for 10 min at 95° C. Subsequently, 40 cycles of PCR were performed. Each cycle consisted of 15 seconds of denaturing (at 95° C.) and 1 min of annealing and extension (at 60° C.) 2^{ΔΔCt} is a ratio of the expression the gene of interest to the reporter18S ribsosome and SP-C messenger RNA (mRNA) levels were normalized to this housekeeping gene.

1.-11. (canceled)

12. A composition for treating an oxygen-induced lung condition comprising
a regenerative mesenchymal stem cell threshold of human mesenchymal stem cells derived from bone marrow or from umbilical cord blood;
a surfactant;
a pharmaceutically acceptable carrier;
one or more stem cell metabolites; and
one or more bioactive factors.

13. The composition of claim 12, wherein the surfactant is selected from the group consisting of a wetting agent, surface tension depressant, detergent, dispersing agent, and emulsifier.

14. The composition of claim 12, wherein the surfactant is selected from the group consisting of surfactant protein A, surfactant protein B, surfactant protein C, surfactant protein D, mixtures and combinations thereof.

15. The composition of claim 12, wherein the pharmaceutically acceptable carrier is selected from the group consisting of phosphate buffer solution (PBS), albumin, sterile saline solution, and perfluorocarbone.

16. The composition of claim 12, wherein the bioactive factor is a member of the TGF-beta superfamily.

17. The composition of claim 12, wherein the bioactive factor is selected from the group consisting of BMP-2, BMP-3, BMP-4, BMP-6 and BMP-7.

18. A method for treating a lung condition comprising the steps of administering a composition comprising:
a regenerative mesenchymal stem cell threshold of human mesenchymal stem cells derived from bone marrow or from umbilical cord blood;
a surfactant;
a pharmaceutically acceptable carrier;
one or more stem cell metabolites; and
one or more bioactive factors to a patient in need thereof; and contacting one or more lung cells with said composition.

19. The method of claim 18, wherein the lung condition is selected from the group consisting of bronchopulmonary dyplasia (BPD), emphysema, and alveolar damage.

20. The method of claim 18, wherein the patient is a premature newborn.

21. The method of claim 18, wherein administering comprises a method of administration selected from the group consisting of intratracheal, aerosol, and nebulized.

22. The method of claim 18, wherein the surfactant is selected from the group consisting of a wetting agent, surface tension depressant, detergent, dispersing agent, and emulsifier.

23. The method of claim 18, wherein the pharmaceutically acceptable carrier is selected from the group consisting of phosphate buffer solution (PBS), albumin, sterile saline solution, and perfluorocarbone.

24. The method of claim 18, wherein the bioactive factor is a member of the TGF-beta superfamily.

25. A method for preventing oxygen induced lung injury comprising the steps of administering a composition comprising:
a regenerative mesenchymal stem cell threshold of human mesenchymal stem cells derived from bone marrow or from umbilical cord blood;
a surfactant;
a pharmaceutically acceptable carrier;
one or more stem cell metabolites; and
one or more bioactive factors to a patient in need thereof; and contacting one or more lung cells with said composition.

26. The method of claim 25, wherein the oxygen induced lung injury is a disease or condition selected from the group consisting of bronchopulmonary dyplasia (BPD), emphysema, cystic fibrosis (CF), acute respiratory distress syndrome (ARDS), pulmonary hypoplasia, and pulmonary hypertension.

27. The method of claim 25, wherein the patient is a premature newborn.

28. The method of claim 25, wherein administering comprises a method of administration selected from the group consisting of intratracheal, aerosol, and nebulized.

29. The method of claim 25, wherein the surfactant is selected from the group consisting of a wetting agent, surface tension depressant, detergent, dispersing agent, and emulsifier.

30. The method of claim 25, wherein the pharmaceutically acceptable carrier is selected from the group consisting of phosphate buffer solution (PBS), albumin, sterile saline solution, and perfluorocarbone.

31. The method of claim 25, wherein the bioactive factor is a member of the TGF-beta superfamily.
